(19) United States
(12) Patent Application Publication
(10) Pub. No.: US 2003/0178195 A1
(43) Pub. Date: Sep. 25, 2003

(54) Method and system for recovery and conversion of subsurface gas hydrates

(76) Inventors: Mark A. Agee, Tulsa, OK (US); Kenneth Ray Roberts, Tulsa, OK (US)

Correspondence Address: GRIGGS BERGEN & JOHNSTON LLP, 2626 COLE AVENUE, SUITE 400, DALLAS, TX, 75204, US

(21) Appl. No.: 10/392,787

(22) Filed: Mar. 19, 2003

Related U.S. Application Data
(60) Provisional application No. 60/365,670, filed on Mar. 20, 2002.

(51) Int. Cl. E21B 43/24; E21B 36/04; E21B 43/26; E21B 43/25; E21B 43/40; C07C 1/12

(52) U.S. Cl. 166/248; 166/267; 166/369; 166/370; 166/308; 166/302; 166/60; 166/279; 166/372; 166/75.12; 166/65.1; 166/357; 518/703; 518/705; 585/801

(57) ABSTRACT

A method and system for the recovery and conversion of subsurface gas hydrates is provided. This is accomplished by accessing a subsurface hydrate formation and treating the formation with a treating system so that gas is released from the hydrate formation. The released gas is then delivered and collected by means of a gas recovery system at a surface location. The gas is converted to liquid hydrocarbons in a conversion system utilizing a synthesis gas unit for producing synthesis gas from the hydrate gas, and a synthesis unit for converting the synthesis gas into liquid hydrocarbons. In at least one embodiment, the synthesis unit utilizes a Fischer-Tropsch reactor. Excess energy produced during the conversion of the hydrate gas can be utilized in the treating and recovery of the hydrate gas.

This application claims priority on U.S. Provisional Patent Application No. 60/365,670, filed on Mar. 20, 2002.

### TECHNICAL FIELD

The invention relates generally to the production of hydrocarbons.

### BACKGROUND

Hydrates are a group of molecular complexes referred to as clathrates or clathrate compounds. Many of these complexes are known and involve a wide variety of organic compounds. They are typically characterized by a phenomenon in which two or more components are associated, without ordinary chemical union, through complete enclosure of one set of molecules in a suitable structure formed by the other. A gas hydrate may be regarded as a solid solution in which the hydrocarbon solute is held in the lattice of the water solvent.

Methane and other hydrocarbons, particularly those light end hydrocarbons, such as ethane, propane and butane, are known to combine with liquid water or ice to form solid compounds that contain both water and individual or mixed hydrocarbons. The gas hydrates resemble ice but remain solid at temperature and pressure conditions above the freezing point of water. They generally consist of about 80 to 85 mol % water and 15 to 20 mol % gas. The gas of most hydrates is predominantly methane, with smaller quantities of other light hydrocarbon gases, such as ethane, propane and butanes. These gas hydrates vary in composition depending upon the conditions. They may be in the form of two crystal structures, referred to as Structure I and Structure II. See, Collett, T. S. and Kuuskraa, V. A., “Hydrates Contain Vast Stores of World Gas Resources,” Oil and Gas Journal, May 11, 1998, pp. 90-95. In the hydrate lattice of Structure I, the hydrate unit cell consists of 46 water molecules that form two small dodecahedral voids and six large tetradecahedral voids that can only hold small gas molecules, such as methane and ethane. In Structure II, the hydrate structure consists of 16 small dodecahedral and 8 large hexakaidechedral voids formed by 136 water molecules. In Structure II, larger gases can be contained within the voids, such as propane and isobutane.

It has been predicted that enormous amounts of hydrocarbon hydrates are located in deposits in various formations throughout the world. These may be found in sediment along the ocean floor, in subsurface deposits below the ocean floor and in onshore subsurface formations located in permafrost regions. It is estimated that as much as 160 to 180 scf of natural gas per cubic foot of hydrate exists in such deposits.

As can be seen, if such hydrates could be effectively and efficiently removed as gas from such formations, a large source of fuel would be available for use. Efforts made to develop methods and equipment for the removal of such hydrates, however, have many shortcomings and appear to have rendered hydrate recovery impractical or uneconomical.

### BRIEF DESCRIPTION OF THE DRAWINGS

For a more complete understanding of the present invention, and the advantages thereof, reference is now made to the following descriptions taken in conjunction with the accompanying figures, in which:

FIG. 1 is a cross-sectional elevational view of a subsurface hydrate-containing formation in association with a free-gas reservoir being accessed to recover hydrate gas;

FIG. 2 is a cross-sectional elevational view of a subsurface hydrate-containing formation where no free-gas reservoir exists, and which is accessed to recover hydrate gas;

FIG. 3 is a schematic representation of a recovery unit used in recovering hydrate gas;

FIG. 4 is a schematic representation of a conversion system used in converting hydrate gas to liquid hydrocarbons; and

FIG. 5 is a flow diagram illustrating a recovery and conversion system for a recovering and converting a given amount of hydrate gas.

### DETAILED DESCRIPTION

Because gas hydrates are solid and exist in reservoirs or formations that are immobile and impermeable, the gas hydrates must be accessed and treated to decompose or dissociate the gas and water forming the hydrate compounds. In U.S. Pat. No. 5,950,732, to Mark A. Agee, et al., which is herein incorporated by reference, a system and method for recovering gas hydrates located on the sea floor is disclosed. Many hydrate formations, however, are located well below the sea floor surface or below permafrost regions located onshore.

It is estimated that subsurface hydrate formations may exist from about 10 to over 1000 meters below the sea floor. Onshore, hydrate formations may be located from about 100 to over 2000 meters below the surface of permafrost regions. Therefore, these formations must first be penetrated or otherwise accessed to enable the hydrates or evolved gases to be removed. The subsurface hydrate formations may include those that may be located beneath at least one generally gas impermeable strata or zone.

Once the hydrate-bearing formation has been accessed, it is then treated to decompose the hydrate to produce gas and water. This may be accomplished by several different techniques, the use of which may vary depending upon the circumstances and particular type of formation to be treated. These techniques include depressurization, thermal stimulation and use of hydrate inhibitors.

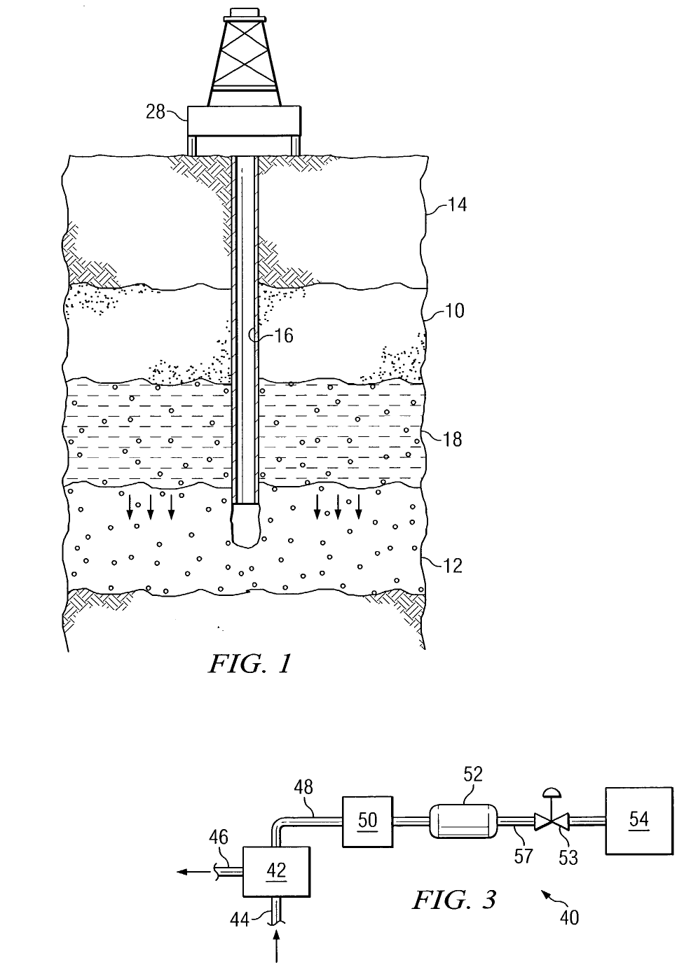

In formations where hydrate is found in conjunction with free gas, depressurization may be the most practical method for recovering gas from the gas hydrate formation. Referring to FIG. 1, here the gas hydrate formation 10, which may be a subsurface offshore or onshore formation, forms a cap or seal above and/or adjacent to a free gas reservoir 12, which may be located in the strata directly beneath or adjacent to the hydrate formation 10. An impermeable strata 14 may be located above the hydrate formation 10, as well. A wellbore 16 is formed that penetrates the formations and communicates with the free gas reservoir for removing and producing gas from the reservoir 12. As the gas is produced from the reservoir 12, the pressure within reservoir 12 is reduced. With this reduction in pressure, the pressure drops below the hydrate equilibrium pressure and causes the adjacent hydrate formation to decompose, forming a dissociation zone 18 of dissociated hydrate gas and water. The resulting gas then enters the gas reservoir, where it is removed through well bore 16.

Depressurization can cause the temperature of the hydrate zone to drop, which can lead to problems with freezing of dissociated water or the reforming of hydrates. It may therefore be desirable to maintain the presence of free gas to sustain the rate of dissociation and maintain production. If free gas is not available, gas lifting methods and water handling may be necessary to continue production from the hydrate zone, which is discussed further on. Depressurization can also be combined with fracturing and other stimulation methods, such as in thermal stimulation or with the use of inhibitors, such as methanol, that are injected into the hydrate zone to facilitate dissociation and to inhibit freezing or refreezing of the dissociated gas and water. Combinations of these techniques may be used as well.

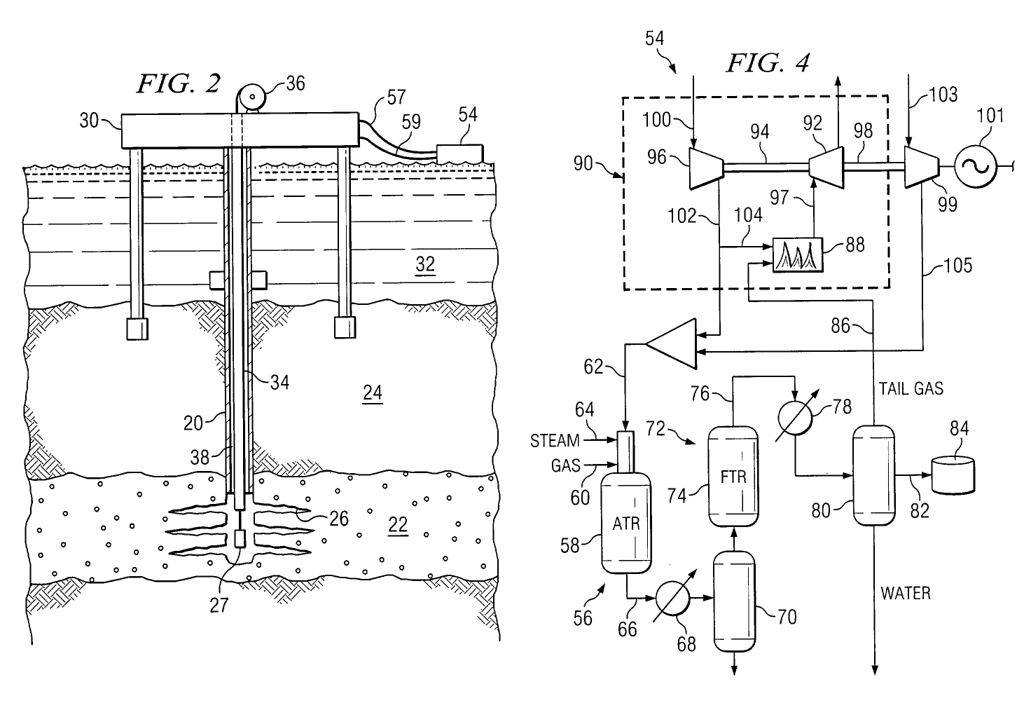

Referring to FIG. 2, one or more well bores, such as the well bore 20, is provided that penetrates a subsurface hydrate-bearing formation 22 where little or no free gas is present adjacent to the hydrate formation. The formation 22 may be a subsurface offshore or onshore formation. An impermeable formation 24, such as impermeable rock or permafrost strata, may be located above the formation 22. The formation 22 may be accessed through conventional drilling techniques, including directional drilling, such as those used for drilling oil and gas wells, and which are well known to those skilled in the art. Directional drilling techniques may be used wherein horizontal or non-vertical boreholes are used to access large areas of the hydrate-bearing formation. Multiple boreholes may also be drilled using directional drilling techniques that offshoot in different or radial directions from a single borehole to access even greater areas of the hydrate-bearing formation. The formation 22 may also be fractured to form fractures 26 utilizing conventional fracturing techniques, well known to those skilled in the art, to thereby increase penetration of and access to the hydrate-bearing formation.

Once the hydrate formation 22 has been penetrated, stimulation or treatment of the hydrate-containing formation can proceed using various techniques. Thermal stimulation may have particular application in situations where no free gas is present, as in the formation of FIG. 2.

In thermal stimulation, heat is provided to the hydrate-containing formation to decompose the hydrate. This may be accomplished by providing heat from the surface through the injection of hot fluids or by generating heat down-hole or in-situ. In the former case, steam, hot water, or hot aqueous saline solution or brine may be injected from the surface into the hydrate formation. A combination of these fluids may also be injected simultaneously or sequentially.

It may be desirable to use brine in many cases as the injection fluid. This is due to the brines' effect as a hydrate inhibitor, which reduces the equilibrium dissociation temperature of the hydrates. As a result, depending upon the salt content of the brine, the reservoir temperature need not to be raised to the same degree as with steam or hot water injection techniques to achieve the same result. Further, the lower dissociation temperature reduces the heat of hydrate dissociation, which results in higher energy efficiency and lower heat loss. Suitable salts for such brines may include NaCl, CaCl₂, MgCl₂ or KCl, as well as others. The particular salt concentration of the brine may depend on the characterization of the hydrate being recovered and the methods used. For instance, hydrate formations formed primarily from methane, which disassociate less readily than hydrates formations formed from methane and other heavier hydrocarbons, may require a higher salt concentration. Further, heating of the brine solution may reduce the need for higher salt concentrations. A particular salinity, however, may be that equal to or greater than that of common seawater.

Pressures and temperatures used in treating the formation may vary. The particular pressure and temperature ranges used may depend upon the in situ temperature and pressure, the composition of the gas hydrate formation, the temperature gradient of the production well, the desired production rate and the method of recovery being employed, as well as other factors.

In offshore applications, seawater provides a naturally abundant supply of saline solution or brine that may be supplemented with additional salts, or water may be evaporated by passing the seawater through a heat exchanger to thereby increase salt concentration, if necessary. It may also be possible to use warm surface seawater without additional heating in certain instances.

Down-hole or in-situ heating may be accomplished by a variety of methods. Electromagnetic heating can be used to heat the formation in situ. This may be either through radio frequency heating techniques or microwave heating. In radio frequency heating, the hydrate formation can be heated at distances from the wellbore that may be too far removed for treatment by hot fluid injection techniques. In radio frequency heating, tubular electrodes, indicated representatively at 27, are inserted into the wellbore. By application of radio frequency energy to these electrodes, heat can be generated in situ to uniformly heat large volumes of the gas hydrates.

Microwave frequency heating can also be used and provides a large volume of heating, away from the wellbore. Microwave heating also evens temperature gradients. In microwave heating, no heat is applied. Instead, microwaves emitted from microwave generator, indicated representatively at 27, pass through the material, with an alternating high frequency electric field. When within this electric field, particles of the material oscillate about their axes, creating intermolecular friction, which heats the material. It is well known that some solids can be heated efficiently in a microwave field as a result of dielectric relaxation, causing the microwaves to act as a transfer agent of the electric power. Microwave heating also has certain advantages. It is possible to create inverse temperature fields due to bulk heating by radiation instead of by conduction, as well as rapid heating, in contrast to the slow heating of conduction. Heating of solids by microwaves is also selective, as gases are essentially “transparent” to microwaves.

It may be desirable to use a combination of electromagnetic heating and thermal injection heating wherein electromagnetic heating is used to melt hydrates around the wellbore, followed by the injection of hot fluids. The electromagnetic heating can provide enough injectivity in the hydrate zones for further stimulation by hot fluid injection techniques.

Another method for downhole or in-situ heating includes the use of an electrical downhole heater, indicated representatively at 27, that is lowered into the wellbore and energized to heat the surrounding formation. It may also be possible to use in-situ combustion as a means for heating the hydrate formation.

Hydrate inhibitors can also be used alone or in combination with any of the above-described techniques. Hydrate inhibitors are injected into the formation and destabilize the hydrates by shifting the hydrate thermodynamic equilibrium. In addition to brine, which was previously discussed with respect to thermal stimulation, other inhibitors include methanol, ammonia and glycol. The concentrations of these inhibitors may vary depending on factors such as the characteristics of the formation being treated and the desired production rate.

A combination of any one or more of the methods previously described may be used to liberate gas from the hydrate formations. Once liberated, the gas is recovered for conversion to liquid hydrocarbons, as is described further below.

Recovery involves transporting the gas to a surface location, such as the onshore recovery station 28 of FIG. 1, or a vessel or platform, such as a tension leg platform, as indicated at 30, located offshore above surface of the body of water 32, in FIG. 2. A variety of techniques may be used to recover gas from the hydrate zone. Generally, however, gas is conducted to the surface by means of the wellbore or a conduit in communication with the wellbore used in penetrating and/or accessing the hydrate-bearing zone. The wellbore or conduit is in fluid communication with the recovery station. The drilling unit used in penetrating the formation may also be located at a remote location several hundred meters from the hydrate formation or the area where the hydrate formation is penetrated through the use of directional or articulated drilling techniques. In this way, dangers encountered in offshore environments due to the instability of the seafloor or lowered buoyancy as a result of evolved gases from the hydrate formation that might endanger a platform or floating vessel can be avoided.

It may be necessary to use gas-lifting methods to bring the gas to the surface. When using depressurization methods where free-gas reservoirs are present, as discussed with respect to FIG. 1, the gas is removed by conventional methods used in producing the free gas. When very little or no free-gas is present or the pressure is insufficient to transport gas to the surface, gas lifting methods may be necessary.

Referring to FIG. 2, an internal liquid delivery conduit 34 is provided within the wellbore 20. During treatment of the hydrate formation, thermal injection fluid, such as the brine or seawater previously discussed, may be introduced through the conduit 34 into the formation. A pump or compressor 36 may be provided for facilitating the injection of such fluids into the formation. Additionally, during recovery, fluid, such as water, gas or air, may be introduced through conduit 34 to facilitate recovery of the hydrate gases. The fluid so introduced causes hydrate gas to be moved upward through the conduit or passage 38 formed by the annular space between the conduit 34 and inner wall of the wellbore 20. The pump or compressor 36 may only need to be operated only at the start of the recover operation, as once the flow of hydrate gas through passage 38 has begun, it may be self-propelled. It may be desirable, however, to continue to operate the pump or compressor 36 to provide fluid flow through conduit 34 to facilitate the speed of removal of the hydrate gases from the formation.

Other methods for transporting the hydrate gases to the surface may be used as well. Methods similar to those described in U.S. Pat. No. 5,950,732 can be used in many instances, particularly in offshore applications.

FIG. 3 shows a recovery unit 40 that may be used at the recovery stations 28 or 30. The recovery unit 40 includes a separator 42 where gas is separated from liquids and solids removed from the wellbore that are received through conduit 44. Liquids and solids removed with the gas are discharged through conduit 46. A three-phase separator may be utilized to facilitate separation of liquids and solids so that these are each discharged through separate conduits, as well. Gas is discharged from the separator 42 through conduit 48. If the gas is wet or contains water vapor, a system (not shown) may be provided for condensing and removing moisture and/or ethane and heavier hydrocarbons. A filtering system 50 may optionally be provided for filtering any entrained particles, if desired, or it may be passed without filtering. The gas can be provided to a storage area 52 prior to being directed to a gas conversion system 54 by means of conduit 57 regulated by valve 53. Optionally, the gas can be directed without storage directly to the gas conversion system 54.

Referring to FIG. 2, in offshore applications, the conversion system 54 may include a floating plant that is coupled to the vessel or platform 30. The conversion system 54 can also provide heated and/or pressurized gas, water, brine or other fluids produced using excess heat or energy produced during the conversion process, as is described below, for injecting into the formation. Such gas or liquids can be directed from the conversion system 54 through line 59 for introduction into the formation, such as through conduit 34.

FIG. 4 shows a schematic of the gas conversion system 54 for converting the hydrate gas to liquid hydrocarbons. The gas conversion system 54 converts the gas recovered from the hydrates into heavier hydrocarbons that may be either liquids or solids, which may be more readily transported. The conversion system also provides excess energy or power that can be used to facilitate hydrate recovery. In this regard, a synthetic production of hydrocarbons using Fischer-Tropsch technology is the desired methodology for conversion of the hydrate gases. Reference is made to U.S. Pat. Nos. 4,883,170; 4,973,453; 5,733,941; 5,861,441; 6,130,259; 6,169,120 and 6,172,124 and U.S. patent application Ser. No. 10/011,789, filed Dec. 5, 2001, all of which are incorporated herein by reference. These patent references set forth the background and technology that may be used as an aspect of the conversion system.

The conversion system 54 includes a synthesis gas generator 56 for producing synthesis gas from the hydrate gas products for conversion to a liquid or solid hydrocarbon (hereinafter “liquid hydrocarbons”). While the following description provides details related to the conversion system, it will be recognized by those skilled in the art that various components, such as valves, heat exchangers, separators, etc., although not specifically described, may be included as part of the conversion system.

The synthesis gas unit 56 may be configured in a number of different ways, but in the embodiment shown, the unit 56 includes a synthesis gas reactor 58 in the form of an autothermal reforming reactor (ATR). A stream of the light hydrocarbon gases produced from the hydrate-bearing zone is introduced into the reactor 58 via line 60. Compressed oxygen-containing gas (OCG) or air is also introduced into the ATR through line 62 to provide a source of O₂ for the necessary reaction. The pressure of the OCG introduced into the ATR may range from about 50 psig to about 500 psig. As used herein, “oxygen-containing gas” shall mean a gas or gas mixture made up of or containing the diatomic form of oxygen or O₂. The OCG or air may be heated in a heat exchanger (not shown). Water (which converts to steam during the reaction) or steam is also introduced along with the gases via line 64. The water may be superheated steam. The ATR may have different forms but generally is comprised of a refractory-lined vessel containing a reforming catalyst, such as a nickel-containing catalyst. The ATR reaction may be adiabatic, with no heat being added or removed from the reactor other than from the feeds and the heat of reaction. The reactions that occur are both exothermic and endothermic with the resulting reactor effluent temperature may range from about 500° F. to about 1000° F. above the feed temperature. The effluent syngas may exit the reactor in the range of from about 1500° F. to about 3000° F., and may be from about 1600° F. to about 2000° F., with a pressure that may range from about 50 to about 500 psig, and may be from about 100 to 400 psig. The conversion system, in particular, may be a hydrocarbon conversion system which utilizes a low-pressure ATR, i.e. at a pressure that may be below 200 psia, or may be below 180 psia. The reaction is carried out under sub-stoichiometric conditions whereby the air/steam/gas mixture is converted to syngas in the form of CO and H₂.

The syngases are discharged through line 66 and may be cooled, typically to a temperature of about 100° F. to about 130° F., by means of heat exchanger 68 before passing to separator 70 to remove free water. Because the reaction is exothermic and there is a large amount of heat generated in the reaction, the heated cooling fluid used for heat exchanger 68 is sufficiently heated for use in other areas, where necessary, such as for use in the hydrate recovery operation, discussed previously. The separator 70 removes moisture from the syngas before it is introduced into the synthesis unit 72. The syngas pressure may be boosted by a syngas booster compressor (not shown). Alternatively, if sufficient pressure exists, the syngas may be delivered without boosting the pressure to the synthesis unit 72.

The synthesis unit 72 includes a Fischer-Tropsch reactor (FTR) 74, which contains a Fischer-Tropsch (F-T) catalyst, such as an iron or cobalt-based catalyst, which may be a supported catalyst, such as a silica, alumina, or silica-alumina supported catalyst. The conditions within reactor 74 are typically maintained at a temperature ranging from about 320° F. to about 600° F. and a pressure of from about 300 psig to about 750 psig. Unlike the ATR, the FTR is not adiabatic. The temperature is controlled in the desired range by removal of heat generated by the Fischer-Tropsch reactions. The heat is typically removed by steam generation within the reactor. Boiler feed water (BFW) is typically delivered to a heat transfer coil (not shown), which is contained within the reaction zone of the FTR to remove the heat of reaction and control the FTR temperature.

Conversion of the synthesis gases to heavy hydrocarbons occurs as they are contacted by the F-T catalyst. The reaction may be represented as follows:

nCO+2nH₂→(—CH₂—)ₙ+nH₂O (1)

The output of the FTR is delivered via line 76 to a heat exchanger 78 and thereafter to separator 80. Because the reaction is exothermic and there is a large amount of heat generated in the reaction, the heated cooling fluid used for heat exchanger 78 can be used in other areas in the conversion system or in the hydrate recovery operation. Within separator 80, heavier liquid hydrocarbons are separated and delivered by line 82 to storage area 84 for later transport and/or further processing (such as hydrocracking, etc.), if necessary. Water, which is produced as a byproduct, is withdrawn through the bottom of separator 80. It may be desirable in some instances to utilize the water withdrawn from separators 70 and 80 in the production of steam for use in other areas or in water-make up in the process.

Tail gas, in the form of light hydrocarbons, nitrogen, etc., is passed through line 86 to a combustor 88. The tail gas of conduit 86 includes nitrogen and other un-reacted substances. While a large variety of tail gas compositions are possible, an example of a tail gas composition ranges may be as follows: carbon monoxide 3-8%, carbon dioxide 3-8%, hydrogen 3-10%, water 0-0.5%, nitrogen 70-90%, methane 1-7%, ethane 0-1%, propane 0-1%, butane 0-1%, pentane+0-1%, each given in volume percent. Additional processing of the residue gases may take place before delivery to the combustor 88. Typically, nitrogen gas will comprise from 70 to 95% by volume of the tail gas and have a low Btu or low heating value. The combustor 88 may therefore be that specifically designed for combusting a low Btu or low heating value fuel, such as the combustor described in U.S. Pat. No. 6,201,029 to Waycuilis, which is herein incorporated by reference.

A gas turbine unit 90 is provided with the conversion system. The gas turbine unit 90 is used to provide power or energy for use in the conversion of the hydrate gases. The gas turbine unit 90 also provides additional power or energy to facilitate hydrate recovery, as will be discussed further on. In preparing a system like system 54, it is preferable to use a gas turbine 90 that is already manufactured by turbine vendors and commercially available and can be used as is or modified within only minimal alterations to accommodate the system.

The gas turbine 90 includes an expander 92, combustor 88, and a compressor 96. The expander 92 is mechanically coupled by a linkage or shaft 94 to the compressor 96. The combustor 88 receives compressed oxygen-containing gas or air through conduit 104 and receives a combustion fuel through conduit 86. The resultant combustion gases are delivered through a conduit 97 to the expander 92 where the resultant power drives shaft 94 to compress air with compressor 96. In addition, the expander 92 may drive the same or a second shaft 98 or other means by which power may be coupled to a second compressor 99, and may also be coupled by another portion of the shaft 98 or separate shaft, or other means of coupling power, to an electrical or mechanical system, such as generator 101. In this way, electrical or mechanical power can be supplied to the conversion or hydrate recovery systems, such as to the compressor or pump 36 of FIG. 2. The second compressor 99 may be an axial-type or centrifugal compressor.

The compressor 96 is used to compress air or an OCG from conduit 100, which may be at ambient conditions. The compressed air is discharged through outlet 102. The compressed air from outlet 102 is split, with a portion being directed to the combustor 88 via line 104 for the combustion of the residue gases previously discussed. Another portion is directed to the ATR via line 62.

The second compressor 99 also receives an oxygen-containing gas, which may be at ambient conditions, such as air or enriched air, through an inlet 103 and compresses the OCG to produce a second compressed oxygen-containing gas feed stream, which is delivered by a conduit 105 to the line 62 for introduction into the synthesis gas unit 56. The second compressor 99 may allow adequate amounts of compressed air to be produced for use in the conversion system without significant modifications or redesigns being made to existing turbines. Examples of suitable commercially available gas turbines include the GE PG9171E gas turbine, manufactured by General Electric, and the G11N2 gas turbine, manufactured by Alstom Power, Baden, Switzerland, each with modifications for extraction of air (i.e., conduit 26, etc.), but other models and makers may be used as well. Compressed air or gases from compressors 96 and 99 can also be diverted for use in hydrate recovery, essentially substituting or serving as the compressor 36 of FIG. 2.

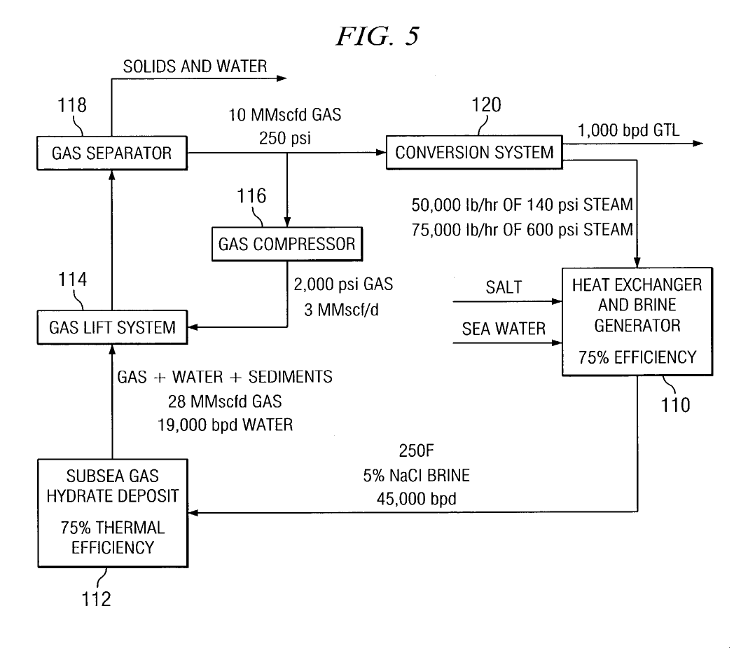

Referring to FIG. 5, a flow diagram illustrating an example of an integrated hydrate recovery and conversion system for a subsurface offshore hydrate formation is disclosed operating at 1000 bpd and utilizing 10 million standard cubic feet of recovered natural gas per day. At this level, 50,000 lb/hr of 140 psi surplus steam and 75,000 of 600 psi surplus steam is generated from the process. This steam would be generated by heat exchangers used in cooling the reaction products of the ATR and FTR, such as exchangers 68 and 78 (FIG. 4), respectively, using water as the cooling fluid.

The steam is used in heat exchanger 110 to heat 45,000 bpd of a 5% by weight NaCl brine solution made from sea water and additional salt, if necessary, at ambient temperature to 250° F. This assumes a 75% efficiency. This is then used to disassociate hydrates of subsurface formation 112 based on the following assumptions:

1. Water depth of 3,500 feet with equivalent pressure of 1700 psi.

2. Ocean bottom temperature equals the hydrate temperature, which is assumed to be 45° F.

3. Hydrate decomposition is 55° F., based upon a 5 wt. % salinity and a pure methane hydrate.

4. The calculated heat of hydrate composition is 15 kcal/gmol of gas (2,700 Btu/lbmole CH₄).

5. Brine injection temperature 250° F.

6. Energy efficiency ratio is 10 to 11, which is defined as the ratio of the heating value of the produced gas to the heat required to decompose hydrates to gas and water.

A gas lift system 114 uses approximately 3 MMscfd of 2000 psi gas from compressor 116. The required compressor BHP is 440 or equivalently 1.1 MMbtu/hr. Lifted gas is provided to separator 118 where solids and liquids are removed. Estimated water production from the hydrate formation is approximately 19,000 bpd, with gas production at 28.5 MMscfd of hydrate gas. Ten million standard cubic feet per day of this is used in the conversion system 120 to thus produce 1000 bpd of liquid hydrocarbon product.

While the invention has been shown in only some of its forms, it should be apparent to those skilled in the art that it is not so limited, but is susceptible to various changes and modifications without departing from the scope of the invention. Accordingly, it is appropriate that the appended claims be construed broadly and in a manner consistent with the scope of the invention.

We claim:

1. A method for recovering and converting gas from subsurface gas hydrates comprising:
accessing a subsurface gas hydrate-containing formation;
treating the accessed gas hydrate-containing formation so that gas is released from the hydrate-containing formation;
collecting the released gas at a surface location; and
converting at least a portion of the collected gas to liquid hydrocarbons at the surface location.

2. The method of claim 1, wherein:
converting at least a portion of the collected gas produces excess energy; and further comprising
utilizing the excess energy from converting the collected gas in treating the accessed gas hydrate-containing formation.

3. The method of claim 1, wherein:
converting at least a portion of the collected gas includes producing a synthesis gas in a synthesis gas unit and converting the synthesis gas to liquid hydrocarbons in a synthesis unit.

4. The method of claim 3, wherein:
the synthesis unit includes a reactor containing a Fischer-Tropsch catalyst and converting the synthesis gas includes contacting the Fischer-Tropsch catalyst with the synthesis gas.

5. The method of claim 1, wherein:
treating the accessed gas hydrate-containing formation includes at least one of depressurization, thermal stimulation and hydrate inhibiter stimulation of the formation.

6. The method of claim 1, wherein:
the gas hydrate-containing formation includes an offshore subsurface formation.

7. The method of claim 1, wherein:
the gas hydrate-containing formation includes an onshore subsurface formation.

8. The method of claim 1, wherein:
the gas hydrate-containing formation is located within a permafrost region.

9. The method of claim 1, wherein:
accessing the subsurface gas hydrate-containing formation includes penetrating and fracturing the formation.

10. A system for recovering and converting gas from subsurface gas hydrates comprising:
a treating system for treating a penetrated gas hydrate-containing formation so that gas is released from the hydrate-containing formation;
a gas recovery system for collecting and delivering released gas from the treated formation to a surface location; and
a conversion system for converting at least a portion of the collected gas to liquid hydrocarbons at the surface location.

11. The system of claim 10, wherein:
the converting system produces excess energy during conversion of the collected gases to liquid hydrocarbons and supplies the excess energy to the treating system.

12. The system of claim 10, wherein:
the converting system includes a synthesis gas unit for converting the collected gas to synthesis gas and a synthesis unit for converting the synthesis gas to liquid hydrocarbons.

13. The system of claim 12, wherein:
the synthesis unit includes a reactor containing a Fischer-Tropsch catalyst and converting the synthesis gas includes contacting the Fischer-Tropsch catalyst with the synthesis gas.

14. The system of claim 10, wherein:
the treating system includes at least one of depressurization unit, thermal stimulation unit and a hydrate inhibiter injector unit.
